(19) United States
(12) Patent Application Publication
(10) Pub. No.: US 2015/0118314 A1
(43) Pub. Date: Apr. 30, 2015

(54) COMPOSITIONS FOR THE PROPHYLAXIS AND TREATMENT OF DERMATOLOGICAL/MUCOSAL DISEASES, AND USES THEREOF

(71) Applicant: DESPHARMA KFT., Budapest (HU)

(72) Inventors: Attila Feleki, Budapest (HU); Theodorus A. C. Migo, Budapest (HU)

(21) Appl. No.: 14/588,655

(22) Filed: Jan. 2, 2015

Related U.S. Application Data
(63) Continuation of application No. 12/737,572, filed on May 11, 2011, now abandoned.

(30) Foreign Application Priority Data
Jul. 28, 2008 (EP) 08462004.6

(51) Int. Cl. A61K 33/06; A61K 9/00; A61K 9/14

(52) U.S. Cl. CPC A61K 33/06; A61K 9/14; A61K 9/0014
USPC 424/489; 424/684

(57) ABSTRACT

A topical composition comprises a clay material with a negative surface charge; magnesium ions and calcium ions; water and/or a non-aqueous solvent; and a substantially non-cationic carrier. The mean particle size of the clay material is at least 5 nm, and the total molar amount of magnesium ions is higher than the total molar amount of calcium ions. The composition is suitable for the prophylaxis and treatment of skin diseases, skin conditions, or mucosal diseases.

### CROSS-REFERENCE TO RELATED APPLICATIONS

This application is a continuation of U.S. patent application Ser. No. 12/737,572, filed on May 11, 2011, which is the National Stage of International Patent Application No. PCT/EP2009/059727, filed on Jul. 28, 2009, which claims priority of European Patent Application No. 08462004.6, filed on Jul. 28, 2008, each of which is incorporated herein by reference.

### FIELD OF THE INVENTION

The present invention relates to compositions for the prophylaxis and treatment of dermatological and mucosal diseases/disorders. In particular, the present invention relates to novel compositions for the prophylaxis and treatment of dermatological diseases/disorders, skin conditions, and mucosal diseases/disorders where epidermal/mucosal barrier formation and/or recovery would have beneficial effects. The invention further relates to the use of such compositions in the manufacture of medicaments/medical devices/care products for the prophylaxis and treatment of said diseases.

### BACKGROUND OF THE INVENTION

The outermost layer of the skin is the epidermis and the mucosal membranes (e.g. buccal, nasal, intestinal, anal) which forms a barrier between the body and the external environment. This semipermeable epidermal barrier prevents both the escape of moisture and the entry of infectious or toxic substances. Common inflammatory skin disorders such as atopic dermatitis, psoriasis and various eczemas exhibit decreased barrier function. Improvement of epidermal barrier function is suggested to improve these skin disorders (The Journal of Clinical Investigation, 116(5): 1150-1158, 2006). In some epidermal/mucous membrane diseases/disorders some major elements for intact barrier function are missing, like e.g. ceramides in case of atopic dermatitis.

Ion gradients of ions such as calcium and magnesium in the epidermis play a crucial role in skin barrier homeostasis. Topical application of calcium solution delayed barrier repair after barrier disruption, however, the application of magnesium salts accelerated barrier recovery (The Journal of Clinical Investigation, 89: 530-538, 1992; Arch. Dermarol. Res., 291:560-563, 1999). Normal skin surface has a negative potential against the inside and the potential decreases immediately after barrier disruption (J. Invest Dermatol., 69:324-327, 1977). Application of external electric potential without topical application of any bioactive chemical materials was reported to improve cutaneous barrier function (J. Invest Dermatol., 118:65-72, 2002).

The effects of topical application of ionic polymers on the damaged skin barrier was evaluated. Application of a nonionic polymer did not affect barrier recovery. Application of sodium salts of anionic polymers accelerated barrier recovery, while that of cationic polymers delayed it. Topical application of a sodium-exchange resin accelerated barrier recovery, but application of a calcium-exchange resin had no effect when the resins had the same structure. (Skin Pharmacol. Physiol., 18:36-41, 2005) Here, according to the authors' conclusion anionic polymer together with magnesium chloride did not support barrier recovery.

Several different treatments of inflammatory skin diseases are known.

WO0185163 suggests either cetyl myristate or cetyl myristate and cetyl palmitate for the treatment or prophylaxis of eczema and/or psoriasis.

DE102007030198A1 suggests aqueous suspensions of silicium dioxide for the treatment of several skin diseases, like atopic dermatitis, psoriasis, inflammation, etc. Preferred embodiments also contain zeolites, like clinoptilolite, fausit, modenit, furthermore calcium carbonate, magnesium carbonate and water. The applied suspension is the carrier of the silicium dioxide particles, and it is either basic (pH of about 9), or acidic (pH of about 3 to 4). Here, calcium and magnesium carbonates are mentioned in some spray and lotion formulation examples, and always in a percentage ratio of 3-3%. When exemplifying capsule, dolomit is mentioned as an ingredient, however, without mentioning the ratio between the calcium and magnesium carbonates. We note here that even same type of dolomites from different geographical originis show significantly different calcium to magnesium ratio. The further problem with dolomite is its nickel content, which is undesired in most of said dermatological diseases. However, the main drawback of these medical formulations is the pH of the formulations that is far from the skin-neutral value (which is about pH 5.5). The strongly basic or acidic character of these formulations might result in irritation and in slower recovery of the skin diseases to be treated. Moreover, one can not reproduce these formulations, since the exact Ca:Mg ratio is not clearly defined in the description. To summarize, the problem with the formulations according to this prior art document is that it either relates to a silicium dioxide carrier with calcium carbonate and magnesium carbonate, however, the pH of these formulations is undesired, or zeolite is mentioned together with dolomit, however, here the calcium to magnesium ratio is not defined.

WO06108414 discloses an agent for use as an oral or topical agent in the therapy and prophylaxis of skin diseases, especially psoriasis, neurodermitis, or also named as atopic dermatitis, where the agent comprises zeolites and stinging nettle leaf extracts or calcium and/or magnesium salts. Although the agent according to this state of the art document comprises calcium and/or magnesium ions, the drawback of this formulation is that it does not necessarily contain both of them.

WO2007022264 discloses a composition for modulating the blood coagulation cascade and assisting in wound healing and body repair. The composition comprises a hemostatically effective amount of a charged oxide, i.e. a silaceous oxide and further comprises a second oxide, such as calcium oxide. Additional components that can be included in the composition are zeolite and/or an inorganic salt, for example a magnesium salt. The magnesium salt is not a compulsory ingredient of the composition. U.S.2007237834 discloses a composition for the treatment of diaper rash, containing divalent metal complexes of zeolite, mainly zinc zeolite.

Inflammatory skin diseases are sometimes associated with bacterial infections. E.g. Staphylococcus aureus superantigens are known to be present in more than 50% of patients with atopic dermatitis and psoriasis, and the severity of these diseases significantly correlated to enterotoxin production of the isolated Staphylococcus aureus strains (Journal of the American Academy of Dermatology, 53:67-72, 2005). Here we have to mention that atopic dermatitis is a hereditary skin symptom, which can be considered as a disease of primary barrier failure.

U.S. Pat. No. 6,551,607 describes a method for sequestration of skin irritants, like superantigens (such as produced by the bacterium Staphylococcus aureus), cytokines, etc. Here, sequestering agent can be adsorbent clays, silicas, etc.

U.S. Pat. No. 5,900,258 describes compositions containing zeolites for preventing microorganisms from growing in a diaper, clothing, bedding, on the skin, etc. This invention was based on the finding according to which the microbial growth is inhibited by zeolites.

Although the role of the epidermal barrier was recognized in several skin diseases/disorders, according to the state of the art there is no pharmaceutical/cosmetic composition available that were optimized to the finding according to which these skin diseases could be treated by epidermal skin barrier formation and/or recovery.

### SUMMARY OF THE INVENTION

The present invention was made in view of the prior art described above, and the object of the present invention is to provide a pharmaceutically and cosmoceutically acceptable composition for the prophylaxis and treatment of dermatological and mucosal diseases/disorders, where epidermal/mucosal barrier formation and/or recovery has beneficial effects.

To solve the problem, the present invention provides a composition containing
a) clay material with a negative surface charge;
b) magnesium ions and calcium ions both either incorporated within the clay material and/or added separately;
c) water and/or non-aqueous solvent;
d) substantially non-cationic carrier; where
i) the mean particle size of the clay material is at least 5 nm; and
ii) the total molar amount of magnesium ions is higher than the total molar amount of calcium ions.

In a preferred embodiment the clay material is a zeolite, more preferably clinoptilolite.

In another preferred embodiment the total molar amount of magnesium ions compared to calcium ions is higher than 1.2× of the total molar amount of calcium ions.

The minimal concentration of magnesium ions is 0.05 weight % and the minimal concentration of calcium ions is 0.04 weight % in the composition of the invention.

In another preferred embodiment, the mean particle size of the clay material is between 5 nm and 100 μm, preferably between 100 nm and 20 μm, more preferably between 1 μm and 10 μm, and most preferably it is about 5 μm to 7 μm.

According to yet another preferred embodiment the separately added magnesium ions are in the form of magnesium chloride or magnesium bromide, preferably magnesium chloride.

According to a further preferred embodiment the pH of the composition is between 5.0 and 7.5, preferably between 5.5 and 6.5 depending on the intended body part to be applied on. In some mucosal disorder treatments pH of the composition can vary between 3 and 7.5.

Yet according to another embodiment, the invention relates to the use of the composition according to the invention for the preparation of a medicament/medical device for the treatment of skin diseases, skin conditions, or mucosal diseases where the formation and/or recovery of epidermal/mucosal barrier function has beneficial effects.

According to a preferred embodiment,
the skin diseases and skin conditions are selected from inflammatory skin diseases, increased fibroblast proliferation, pruritus, physical damage of the skin surface, xerosis, bruises, hyperproliferation states of the skin, transepidermal water loss, wound, presence of positively charged or polaric superantigens, like e.g. bacteria with a positive surface charge; and
mucosal diseases are selected from aphtous stomatitis, other oral ulcers, destroyed barrier by misuse of the toothbrush, other injuries of mucosal membranes, gingivitis, diseases of the nasopharyngeal or other mucosa, wound and presence of positively charged or polaric superantigens, like e.g. bacterial antigens with a positive surface charge.

According to a more preferred embodiment, the inflammatory skin disease is psoriasis, atopic dermatitis, or various types of eczema.

Since some of the skin disorders are not recognized as diseases, but rather as cosmetic problems, the composition according to the present invention is useful also in cosmetology, not only in dermatology. In other words, the term “medicament” in the present specification shall be understood beside being a product useful in medicine, also as a product useful in cosmetology.

The compositions of the present invention are also useful for the prophylaxis of the above mentioned diseases and conditions.

According to another preferred embodiment, the medicament is in the form of a cream, lotion, gel, ointment, cutaneous solution, suspension, spray, powder, foam, bath additive, film forming agent, collodion, impregnated dressing or medicated plaster, mouth wash, and nasal spray.

We have surprisingly found that a composition that is able to form an electric double layer in aqueous or non-aqueous medium, and shows a stable negative charge, has a cation exchange capacity and cation binding capacity, furthermore contains total magnesium ions more than the total amount of calcium ions, in combination with a non-ionic, anionic, amphoteric or very weakly cationic carrier, is able to recover damaged epidermal barrier upon topical application on the affected skin area.

A negative charge can be formed by any natural or artificial clay material with a negative surface charge. Zeolites, especially clinoptilolite are the preferred choice of clay material according to the present invention. The clay material is preferably in the form of small particles (micronized particles), since this average size range provides acceptable small particles to be applicable on skin surface, and acceptable high specific surface and charge. According to the present invention the minimal average clay particle size is 5 nm,—the applied particle size depends on the surface type and the status of its barrier damage—, which can make sure that the composition does not penetrate through the skin/into the blood stream, therefore can remain on the skin surface for a time period which is necessary for the desired therapeutic effect.

Magnesium ions play a crucial role in the epidermal/mucosal barrier function, however, calcium ions are found to be against its proper function. Accordingly, it is necessary to include a calculated amount of magnesium ions compared to calcium ions being present in the composition according to the present invention. The molar amount of magnesium ion to calcium ion ratio should be higher than 1.0. Preferably there are 1.2× more magnesium ions in the composition than calcium ions. Magnesium ions can be introduced into the composition according to the invention either as part of the applied clay material, and/or separately, as any physiologically acceptable magnesium salt.

The presence of calcium ions in the composition of the invention is also necessary. Calcium assists exocytosis of the lamellar granules in stratum granulosum and induces terminal differentiation of stratum corneum, formation of cornified envelope and also epidermal lipid synthesis. After extrusion of lamellar granules content, the applied ionic environment contribute for the stacks of membranes to reorganize structurally, to form patterned lamellar sheets via edge-to-edge fusion of the lipid stacks. Calcium also activates the trans-glutaminase enzyme to irreversibly cross-link the cornefied envelope proteins, creating a tough insoluble sac that surrounds the keratin fibers.

Although some of the prior art documents mention both magnesium and calcium ions as possible ingredients of medicaments for certain skin diseases, or mention additives that contain undefined amount of calcium and magnesium ions (e.g. stinging nettle leaf extracts or dolomites), we surprisingly found that the magnesium to calcium ion ratio should be well defined for the desired therapeutic effect. Accordingly, the present invention provides a composition where the total molar amount of magnesium ions is higher than the total molar amount of calcium ions. The phrase total molar amount is used to refer cations both from the clay material and to separately added salts. Since compositions according to the state of the art contain only additives where magnesium to calcium ratio is undefined (i.e. calcium could be present even in higher amount than magnesium), the desired therapeutic effect can not necessarily be achieved by them.

According to the present invention, the composition should be able to form an electric double layer in aqueous or non-aqueous medium, and should provide a stable negative charge.

The pH of the composition according to the present invention should be close to the pH of the healthy skin surface, which is about pH 5.5. Furthermore, the desired negative electric charge can be achieved around this pH with the composition of the present invention. If the composition is for the treatment of mucosal diseases, the pH should be close to the pH of the certain healthy mucosa. Accordingly, a composition to be applied on skin surface has preferably a pH from 5 to 7, and the pH of a composition to be applied on mucosal surface vary from 3 to 7.5 depending on the certain mucosa and disorder type.

The composition according to the present invention also necessarily contains a substantially non-cationic, i.e. non-ionic, anionic, amphoteric or very weakly cationic carrier. Since the present invention aimed at providing a composition forming a negative net charge, only substantially non-cationic carriers could be acceptable. On the other hand compositions containing slightly cationic carriers are also within the scope of the present invention, if the total net charge of the composition remains negative.

We have found that the above detailed composition according to the present invention is useful for preparation of medicaments/medical devices for treating such diseases, where the formation and/or recovery of epidermal/mucosal barrier function has beneficial effects, since these medicaments/medical devices improve disrupted epidermal/mucosal barrier function. (The term “medical device” within the present specification means a pharmaceutical or cosmetical product where a primary biophysical effect is thought to be the basis of the effectiveness of the product.) Several skin diseases/disorders are known to be associated with malfunction of the epidermal barrier, e.g. inflammatory skin diseases (like psoriasis, atopic dermatitis, or various types of eczema), increased fibroblast proliferation, pruritus, physical damage of the skin surface, xerosis (hyperproliferated scaly skin caused by dry enviroment), bruises, hyperproliferation states of the skin, and transepidermal water loss, wound healing. Mucosal membrane barrier disorders are e.g. aphtous stomatitis, bruises caused by e.g. tooth brush usage, wounds, ulcers, mucosal disruptions caused by chemical/biological agents (like bacterial and viral agents).

Since recovery of the damaged epidermal/mucosal barrier is a longer process, it is necessary that the medicament of the present invention remains on the affected skin/mucosa area as long as possible. Therefore, the form of the medicament according to the present invention should be able to keep the medicament on the skin surface for a longer time. Accordingly, cream, lotion, gel, ointment, cutaneous solution, suspension, spray, powder, foam, film layer (i.e. a film forming agent), bath additive, collodion, impregnated dressing or medicated plaster, mouth wash, and nasal spray are preferable from this point of view. Soaps or shampoos are less preferred, however, are also within the scope of the present invention.

### DETAILED DESCRIPTION OF THE INVENTION

Magnesium ions (in the form of MgCl₂) are historically documented having effect on regulation of fibroblast replication, barrier recovery acceleration, supressing effect on skin's Langerhans cells, which leads to lowering antigen presentation capacity of said cells.

Clinoptilolite (Ca-K-Mg-Na-zeolite), one of the most preferred zeolite according to the present invention, dispersed in water or in other non-aqueous solvent, shows a strong negative surface charge, that is formed as an electric double layer around the clay material particles, which bind cations. Accordingly, a strong negative charge is formed in the dispersion medium. Zeta potential measurements were used for quantification of the magnitude of the electrical charge at the double layer. Any natural or artificial clay material with a negative surface charge is useful as the clay material according to the present invention, for example, bentonite, montmorillonite, beidelite, hectorite, saponite, stevensite, mordenit, laumonite, phillipsit, heulandit, stilbit, kabazit, stellerit, or laponite.

The clay material, especially the zeolite has the following beneficial effects regarding epidermal/mucosal barrier formation and/or recovery:
i) the boost of negative electric charge;
ii) the cation exchange; and
iii) the cation binding capacity.

These three effects trigger the epidermal/mucosal barrier formation and/or recovery.

Furthermore, after application of the composition of the invention on the desired skin/mucosa surface, a layer is formed that acts also as a molecular sieve. This keeps away bacterias from the said surface, however, it enables for the skin/mucosa to function properly.

One of the main advantage of using zeolites, especially clinoptilolite in the composition according to the invention is that it shows a synergic effect together with magnesium ions. Namely, binding Mg²⁺ ions by the clinoptilolite is increasing the bioavailability of magnesium ions by the skin.

However, we found that using zeolites and magnesium salts exclusively, as powder on dry surface will not lead to epidermal barrier recovery effects. But when using also a humectant (a hygroscopic substance, increasing water content capacity of the skin) rich emollient as said carrier, including water, to moisturize the skin, this will strongly initiate barrier recovery and prevent transepidermal water loss.

Accordingly, when using the above components, namely a clay material of a mean particle size of more than 5 nm, preferably more than 50 nm, preferably zeolite, more preferably clinoptilolite, furthermore magnesium ions and calcium ions, water and a substantially non-cationic carrier in a single composition, a surprisingly increased epidermal barrier recovery effect could be achieved, because of the improvement/formation of the stable negative potential on the affected skin surface, furthermore because of the cation exchange and cation binding capacity, magnesium supply and moisturizing of the skin occurs at the same time, all these effects support epidermal barrier recovery, made by one single composition according to the present invention.

We identified surprisingly an additional beneficial effect of the composition of the present invention. Namely, a negative charge and the clay material together is able to eliminate positively charged superantigens, bacterial antigens or even bacteria from the skin. Since some of the skin/mucosal disorders are associated with bacterial infections (see above) this effect also increases the efficacy of the composition in the treatment of some of said skin diseases.

Any magnesium and calcium salt that are physiologically acceptable and does not impair negative zeta potential of the composition can be used. Both inorganic, or organic magnesium and calcium salts could be acceptable, namely magnesium and calcium salts of chloride, bromide, sulphate, phosphate, acetate, citrate, etc. Preferred magnesium and calcium salt is chloride or bromide. It is obvious for the person skilled in the art that magnesium and calcium salts can be used in the form of various hydrates. According to a most preferred embodiment of the present invention magnesium chloride hexahydrate is used as separately added magnesium source of the composition. The amount of the magnesium chloride hexahydrate can vary from about 0.02 w/w % to about 90 w/w %, preferably from about 0.2 w/w % to about 30 w/w % of the composition.

The composition of the present invention can also contain pharmaceutically acceptable additives. For example vitamins, especially dermatologically preferred vitamins, or even plant extracts could be added to the composition. Preservatives (e.g. methylparaben, propylparaben, butylparaben, diazolidinyl urea, sorbic acid, etc.), pH adjusting additives (sodium hydroxide, hydrogen chloride), moisturizers (e.g. urea, gold colloids, silver colloids, ceramides, etc), pigments (iron oxide) that are known according to the state of the art can also be applied. Other pharmaceutically acceptable additives can also be applied according to the present invention, like glyceryl monostearate, cocoglycerides, glyceryl stearates, cetyl alcohol, isopropyl myristate, ceteareth-20, ceteareth-12, cetyl palmitate, etc.

The composition can also contain other active ingredients that are useful in the prophylaxis and treatment of the dermatological disease or skin conditions to be treated. For example ceramide, ceramidase antagonists, or ceramide production agonists can be added to the composition in the treatment of atopic dermatitis, since this disease is characterized by low ceramide levels. For the treatment of psoriasis the addition of vitamin D analogs and/or steroids to the composition could be advantageous. Other examples for active ingredients in compositions for the treatment of psoriasis can include tar products and natural herbal extracts, like e.g. calendula. For the treatment of different types eczema the addition of various types of peptides could be useful. Also wound healing additives are useful in some of the above mentioned disease, here zinc-hyaluronate, silver or gold colloides are worth mentioning. Regulators of the inflammatory cascade elements which can effect barrier damage recovery (like e.g. interleukins 4, 8 or 10) can also be applied as additional active ingredients.

The optimal molar amount of the applied magnesium ions is dependent on the skin disorder to be treated. Namely, composition for the treatment of atopic dermatitis have lower amount of magnesium ions, since the treatment of an open wound on children can not be treated with compositions with high salt-contents because of irritation. However, compositions for the treatment of psoriasis should have higher magnesium ion concentration.

As mentioned before, the form of the medicament according to the present invention can be any formula able to keep the medicament on the skin surface for a longer time. Accordingly, cream, gel, lotion , ointment, cutaneous solution, suspension, spray, foam, bath additive, collodion, film forming agent, impregnated dressing or medicated plaster are preferred formulas. In respect of mucosal application also powder, film forming solution, mouth wash and nasal spray are preferred.

The solvent of the composition (i.e. component “c” above and in claim 1) can be either aqueous or non-aqueous. Since one of the main characteristics of the composition of our invention is that it forms an electric double layer in the medium and provide a negative electric potential change on the applied surface, any solvent can be used, as the medium of the composition, which allows to form said electric double layer. Accordingly, the solvent of the composition of the present invention can be selected from the group of the following solvents: water, ethanol, glycerol, natural or artificial fats and oils, and mixture thereof, e.g. water/ethanol, oil-in-ethanol. Also emulsions and suspension can form the medium of the composition of the invention.

Cream could be either oil-in-water or water-in-oil type. According to the present invention oil-in-water type cream is preferred. Several emulgators are acceptable for the purpose of the present invention, like e.g. alkyl sulphates, alkyl amines, alkyl pyrimidin compounds, etc.

Also there are several acceptable oils for cream formulation, like e.g. white petrolatum, paraffin, cetearyl alcohol, cocoglycerides, cetyl alcohol, isopropyl miristate, cetyl palmitate, butyrum cacao, oleum helianthi, cera alba, lanolin, isopropyl palmitate, stearic acid, magnesium stearate.

For preparation of a gel, the following gel forming additives could be used for example: cellulose gum (carboxymethyl cellulose), hydroxypropyl cellulose, methylcellulose, hydroxyethyl cellulose, ethylhydroxy cellulose, or laponite. Solvent/dispergating medium for a gel is preferably a water/glycerol or water proylenglycol mixture.

Zeta potential shows the inherent electric characteristic of the composition according to the invention. Briefly, zeta potential is the electrokinetic potential in colloidal systems, namely zeta potential is electric potential in the interfacial double layer at the location of the slipping plane versus a point in the bulk fluid away from the interface, i.e. the potential difference between the dispersion medium and the stationary layer of fluid attached to the dispersed particle. Since the healthy skin surface has a negative charge, the composition according to the invention shall be able to form an even more negative environment on the surface of the skin,a negative charge boost to trigger barrier repair cascading mechanisms. For this purpose, the composition should show a stable negative zeta potential, which can be measured during manufacture of the medicament of the present invention.

### EXAMPLES

Hereinafter, the present invention is described in more detail and specifically with reference to the Examples, which however are not intended to limit the present invention.

### Example 1a

### Cream Formulation for the Treatment of Atopic Dermatitis

A cream was formulated for the treatment of atopic dermatitis, where said cream consisted of the following ingredients (INCI (International Nomenclature of Cosmetic Ingredients) names of the ingredients are provided):

| Ingredient | m/m % |
|---|---|
| Aqua | 57.600 |
| White petrolatum | 17.153 |
| Cetearyl alcohol | 10.292 |
| Paraffin | 3.430 |
| Polysorbate 60 | 3.430 |
| Clinoptilolite* | 3.439 |
| Glycerine | 2.579 |
| Alcohol | 1.543 |
| Magnesium chloride | 0.214 |
| Methylparaben | 0.171 |
| Phospholipid | 0.133 |
| Sphyngolipid | 0.016 |

*contains calcium

### Example 1b

### Zeta Potential Measurement

Zeta potential of the cream according to Example 1a was measured. Measurement was performed by Brookhaven ZetaPALS instrument in the presence of 1 mM MgCl₂ inert electrolyte at 30° C. The instrument is equipped with Peltier element for the active temperature control. The ZetaPALS determines zeta potential using Phase Analysis Light Scattering method based on the shifted frequency spectrum. Palladium electrodes with acrylic supports were used for the measurement.

Results of the zeta potential measurement:

Conductivity: 566 μS

Electroforetic mobility: −0.87* 10⁻⁸m²/Vs

Zeta potential (calculated by Smoluchowsky theory): −10.02 mV

Zeta potential (calculated by Hückel theory): −15.04 mV

Accordingly, zeta potential of the composition is within the required negative range.

### Example 1c

### Electric Surface Potential Measurements

Electric surface potential measurements were carried out with the cream of Example 1a on intact skin surfaces of human volunteers.

Measurement conditions:
Machine: Consort C831 electrochemical analysator
Electrodes: EKG adhesive electrodes
Connecting medium: REXTRA EKG, EEG, EMG diagnostic gel for medical use, pH 6.5
Ingredients: Aqua, Carbomer, Triethanolamine, Methylchloroisothiazolinone, methylisothiazolinone
Measured data row: 25 points, 10 sec/point
Place of measurements: lower arm flexor area in inactive position
Cream applied on: a 5×5 cm surface, after 15 minutes cream removed
Distance of the electrodes: 10 cm
Number of volunteers: 3

Results are shown in the following Table 1.

TABLE 1
Results of the electric surface potential measurements of Example 1c

| | | Volunteer 1 | | | Volunteer 2 | | | Volunteer 3 | | |
|---|---|---|---|---|---|---|---|---|---|---|
| Meas. point | sec | M1 | M2 | result | M1 | M2 | result | M1 | M2 | result |
| 1 | 0 | −65 | −122 | −57 | 20 | −86 | −106 | 33 | −98 | −131 |
| 2 | 10 | −60 | −127 | −67 | 20 | −90 | −110 | 41 | −99 | −140 |
| 3 | 20 | −55 | −133 | −78 | 21 | −94 | −115 | 43 | −101 | −144 |
| 4 | 30 | −53 | −138 | −85 | 28 | −98 | −126 | 45 | −103 | −148 |
| 5 | 40 | −52 | −145 | −93 | 21 | −102 | −123 | 45 | −105 | −150 |
| 6 | 50 | −51 | −151 | −100 | 21 | −108 | −129 | 44 | −108 | −152 |
| 7 | 60 | −47 | −158 | −111 | 22 | −110 | −132 | 45 | −11 | −56 |
| 8 | 70 | −40 | −165 | −125 | 22 | −114 | −136 | 44 | −114 | −158 |
| 9 | 80 | −36 | −172 | −136 | 23 | −117 | −140 | 45 | −118 | −163 |
| 10 | 90 | −34 | −178 | −144 | 15 | −121 | −136 | 46 | −122 | −168 |
| 11 | 100 | −31 | −183 | −152 | 21 | −125 | −146 | 45 | −128 | −173 |
| 12 | 110 | −28 | −188 | −160 | 22 | −129 | −151 | 46 | −129 | −175 |
| 13 | 120 | −21 | −192 | −171 | 23 | −133 | −156 | 46 | −133 | −179 |
| 14 | 130 | −18 | −196 | −178 | 23 | −138 | −161 | 47 | −136 | −183 |
| 15 | 140 | −17 | −199 | −182 | 23 | −143 | −166 | 48 | −139 | −187 |
| 16 | 150 | −17 | −203 | −186 | 25 | −146 | −171 | 49 | −143 | −192 |
| 17 | 160 | −16 | −205 | −189 | 23 | −150 | −173 | 49 | −145 | −194 |
| 18 | 170 | −17 | −208 | −191 | 23 | −153 | −176 | 49 | −147 | −196 |
| 19 | 180 | −16 | −211 | −195 | 24 | −157 | −181 | 50 | −149 | −199 |
| 20 | 190 | −15 | −213 | −198 | 23 | −160 | −183 | 50 | −152 | −202 |
| | | −34 | −174 | −140 | 22 | −124 | −146 | 46 | −119 | −165 |

| avarage | M1 | M2 | result |
|---|---|---|---|
| | 11 | −139 | −150 mV |

where
M1: potential difference between 2 points with connecting medium;
M2: potential different between 2 points with connecting medium and cream of Example 1a;
Result: surface electric potential difference between M1 and M2 in mV.

These results show a definite effect on the electric potential of the skin surface.

### Example 2

### Cream Formulation for the Treatment of Psoriasis

A cream was formulated for the treatment of psoriasis, where said cream consisted of the following ingredients (INCI (International Nomenclature of Cosmetic Ingredients) names of the ingredients are provided):

| Ingredient | m/m % |
|---|---|
| Aqua | 41.81 |
| Magnesium Chloride | 17.22 |
| White petrolatum | 14.22 |
| Cetearyl Alcohol | 8.59 |
| Zeolite* | 8.00 |
| Paraffin | 2.87 |
| Polysorbate 60 | 2.86 |
| Gylcerin | 3.00 |
| Alcohol | 1.29 |
| Methylparaben | 0.14 |

*zeolite stands for clinoptilolite

### Example 3

### Cream Formulation for the Treatment of Psoriasis

A cream was formulated for the treatment of psoriasis on the scalp, where said cream consisted of the following ingredients (INCI (International Nomenclature of Cosmetic Ingredients) names of the ingredients are provided):

| Ingredient | m/m % |
|---|---|
| Aqua | 86.86 |
| Magnesium Chloride | 4.00 |
| Glycerin | 4.95 |
| Cellulose Gum | 1.19 |
| Zeolite* | 2.00 |
| Propylene Glycol | 0.55 |
| Diazolidinyl Urea | 0.30 |
| Methylparaben | 0.12 |
| Propylparaben | 0.03 |

*zeolite stands for clinoptilolite

### Example 4

### Gel Formulation

A gel containing the composition of the present invention was formulated with the following ingredients:

| Ingredient | m/m % |
|---|---|
| Aqua | 83.81 |
| Magnesium Chloride | 7.92 |
| Glycerin | 4.94 |
| Cellulose Gum | 1.18 |
| Zeolite* | 2.00 |
| Methylparaben | 0.15 |

*zeolite stands for clinoptilolite

The water was heated to 75° C., methlyparaben is dissolved in the water. The mixture is cooled to room temperature, and magnesium chloride was added. The zeolite was suspended in the glycerol, and this suspension was added to the aqueous solution of the magnesium chloride under stirring. Cellulose gum was added and the mixture was stirred until formation of the final gel structure.

### Example 5

### Gel Formulation

A gel containing the composition of the present invention was formulated with the following ingredients:

| Ingredient | m/m % |
|---|---|
| Aqua | 77.12 |
| Magnesium Chloride | 7.92 |
| Propylenglycol | 9.90 |
| Hydroxypropyl Cellulose | 1.97 |
| Zeolite* | 2.00 |
| Alcoholum 96% | 0.99 |
| Methylparaben | 0.10 |

*zeolite stands for clinoptilolite

Magnesium chloride was dissolved in the water. The zeolite was suspended in the propylenglycol, and added under continuous stirring to the aqueous phase. Hydroxyprpyl cellulose was added to the aqueous phase until formation of the final gel structure. Finally, the methylparaben dissolved in the ethanol was added slowly into the gel.

### Example 6

### Gel Formulation

A gel containing the composition of the present invention was formulated with the following ingredients:

| Ingredient | m/m % |
|---|---|
| Aqua | 71.92 |
| Magnesium Chloride | 7.83 |
| Glycerol | 2.94 |
| Propylenglycol | 11.76 |
| Cellulose Gum | 0.49 |
| Methylcellulose | 1.96 |
| Zeolite* | 3.00 |
| Methylparaben | 0.10 |

*zeolite stands for clinoptilolite

The water was heated to 75° C., methlyparaben is dissolved in the water. The mixture is cooled to room temperature, and magnesium chloride was added. The zeolite was suspended in the glycerol, and this suspension was added to the aqueous solution of the magnesium chloride under stirring. Cellulose gum and methylcellulose was added and the mixture was stirred until formation of the final gel structure.

### Example 7

### Cream Formulation

A cream containing the composition of the present invention was formulated with the following ingredients:

| Ingredient | m/m % |
|---|---|
| Aqua | 45.14 |
| Magnesium Chloride | 18.61 |
| White petrolatum | 15.39 |
| Cetearyl alcohol | 9.24 |
| Zeolite* | 4.00 |
| Paraffin | 3.00 |
| Polysorbate 60 | 3.08 |
| Alcohol | 1.39 |
| Methylparaben | 0.15 |

*zeolite stands for clinoptilolite

Water was heated to 75° C. and magnesium chloride was dissolved in it, followed by dispersion of the zeolite. White petrolatum, cetearyl alcohol, paraffin and polysorbate 60 were mixed in a separate flask and heated to 75° C. Oil phase was poured to the water phase, emulsified, cooled under continuous stirring, and finally methylparaben dissolved in alcohol was added.

### Example 8

### Cream Formulation

A cream containing the composition of the present invention was formulated with the following ingredients:

| Ingredient | m/m % |
|---|---|
| Aqua | 49.25 |
| Magnesium Chloride | 18.61 |
| White petrolatum | 15.39 |
| Cetearyl alcohol | 9.24 |
| Zeolite* | 1.20 |
| Isopropyl miristate | 3.08 |
| Polysorbate 60 | 3.08 |
| Methylparaben | 0.10 |
| Propylparaben | 0.05 |

*zeolite stands for clinoptilolite

Water was heated to 75° C. and methylparaben and propylparaben was dissolved in it, and zeolite was dispersed in this solution. White petrolatum, cetearyl alcohol, isopropyl myristate and polysorbate 60 were mixed in a separate flask and heated to 75° C. Oil phase was poured to the water phase, emulsified, cooled under continuous stirring, and finally magnesium chloride dissolved in some part of the water was added.

1. A topical composition containing
a) zeolite clay material with a negative surface charge;
b) magnesium ions and calcium ions;
c) water and/or non-aqueous solvent;
d) substantially non-cationic carrier; and
e) a humectant;
the clay material having a mean particle size in the range of about 5 nm to about 100 μm; and
molar ratio of magnesium ions to calcium ions being greater than 1.

2. Composition according to claim 1, where the total molar amount of magnesium ions compared to calcium ions is more than 1.2 times the total molar amount of calcium ions.

3. Composition according to claim 1, where the mean particle size of the clay material is in the range of 100 nm to 20 μm.

4. Composition according to claim 1, containing separately added magnesium ions in the form of magnesium chloride or magnesium bromide.

5. Composition according to claim 1, where the pH of the composition is in the range of 3.0 to 7.5.

6. Composition according to claim 1 where the clay material is clinoptilolite.

7. Composition according to claim 1, where the mean particle size of the clay material is in the range of 1 μm to 10 μm.

8. Composition according to claim 1, where the mean particle size of the clay material is in the range of 5 μm to 7 μm.

9. Composition according to claim 1, where the pH of the composition is in the range of 5.0 to 7.5.

10. Composition according to claim 1, where the pH of the composition is in the range of 5.5 to 6.5.
